(19) United States
(12) Patent Application Publication
(10) Pub. No.: US 2026/0232402 A1
(43) Pub. Date: Aug. 13, 2026

(54) A HOLDING DEVICE FOR MEDICAL EQUIPMENT

(71) Applicant: FHS GROUP HOLDINGS PTY LTD, Hamilton (NZ)

(72) Inventor: Brian Max DONELLEY, Hamilton (NZ)

(73) Assignee: FHS GROUP HOLDINGS PTY LTD, Hamilton (NZ)

(21) Appl. No.: 19/156,258

(22) PCT Filed: Feb. 15, 2024
(86) PCT No.: PCT/NZ2024/050016

§ 371 Date: Aug. 13, 2025

(30) Foreign Application Priority Data
Feb. 16, 2023 (NZ) 796979

(51) Int. Cl. A61B 50/20; F16M 13/02

(52) U.S. Cl. CPC A61B 50/20; F16M 13/02

(57) ABSTRACT

The invention relates to a holding device for supporting medical equipment encased or partially encased in packaging, the packaging being configurable from an unopened state to an opened state, the holding device comprising a body shaped to receive and hold the medical equipment, one or more retaining portions and a mounting component. One or more of the retaining portions are adapted to retain at least a portion of the packaging when configured to the opened state, such that upon or during removal of the medical equipment from the holding device and/or the opened packaging, the packaging remains retained or partially retained by the retaining portions of the holding device.

### FIELD OF THE INVENTION

The present invention relates to a holding device for medical equipment.

### BACKGROUND OF THE INVENTION

Medical equipment that is expected come into contact with patients should remain as sterile and/or clean as possible to prevent infection. For this reason, most medical equipment comes sealed and sterilised in individual packaging.

There are instances where medical equipment may be required urgently. For example if there is an obstruction or potential obstruction in a patient's airway, or potential for inhalation of oral or gastric contents (typically saliva, blood or vomit), or having a clear view of the anatomy-unobstructed by oral or gastric contents (typically a prerequisite for intubation). All these instances can be potentially life threatening. A yankauer sucker is ideally deployed with as little delay as possible to clear a patient's airway and thus prevent these life-threatening complications. For this reason, packaging of sterilised medical equipment is sometimes opened long before it is required to provide quick access to the sterilised medical equipment. As such, the medical equipment should be stored in a way that decreases risk of contamination.

Further, if the medical equipment is a suction apparatus, the pre-opened packaging may remain attached or otherwise bound to the suction apparatus due to suction and may therefore require additional effort and time to remove the pre-opened packaging before deploying the suction apparatus. It would therefore be useful to facilitate quick access to medical equipment while minimising risk of contaminating the equipment in pre-opened packaging.

Reference to any prior art in the specification is not, and should not be taken as, an acknowledgment or any form of suggestion that this prior art forms part of the common general knowledge in any jurisdiction or that this prior art could reasonably be expected to be understood, regarded as relevant and/or combined with other pieces of prior art by a person skilled in the art.

### SUMMARY OF THE INVENTION

As used herein, except where the context requires otherwise, the term “comprise” and variations of the term, such as “comprising”, “comprises” and “comprised”, are not intended to exclude further additives, components, integers or steps.

According to a first aspect of the invention, there is provided a holding device for supporting medical equipment encased or partially encased in packaging, the packaging being configurable from an unopened state to an opened state, the holding device comprising;
a body shaped to receive and hold the medical equipment;
one or more retaining portions; and
a mounting component;
wherein the one or more retaining portions are adapted to retain at least a portion of the packaging when configured to the opened state, such that upon or during removal of the medical equipment from the holding device and/or the opened packaging, the packaging remains retained or partially retained by the retaining portions of the holding device.

In one example the body is a hollow body formed from one or more walls defining a central cavity, the central cavity shaped to receive and hold the medical equipment.

In further examples the body is a cylindrical hollow body.

In further examples the hollow body is open at a first end and at an opposing second end.

In further examples at least a portion of the hollow body includes an inner diameter sized to retain a packaged medical equipment.

In further examples the one or more retaining portions are in or on the one or more walls of the hollow body.

In further examples the holding device comprises two retaining portions positioned on opposing sides of the hollow body.

In further examples the hollow body comprises at least one aperture in the body wall, the aperture defining a space the surrounds at least a portion of a retaining portion.

In further examples the holding device includes two opposing elongate apertures in the body walls, the openings extending between 10%-90% of the length of the hollow body.

In further examples the apertures extend between 40%-60% of the length of the hollow body.

In further examples the retaining portion(s) comprise a tongue having a top end and an opposing base end, the tongue extending at least partially outwardly from the hollow body wall at a top end of the tongue to define a space or gap between the top end of the tongue and hollow body wall, the tongue directly or indirectly mounted to the hollow body wall at a base end of the tongue.

In examples, the at least one aperture surrounds or partially surrounds a curved tongue, the tongue directly or indirectly connected to the hollow body at a/the base end of the tongue.

In further examples the tongue(s) include a flanged and/or curved tip at the top end of the tongue.

In further examples the tongue(s) may be tapered or curved at one or more regions or along one or more axis.

In further examples the tongue(s) is curved such that a portion of the curved tongue extends through the opening into the central cavity of the hollow body.

In further examples the device includes two opposing tongue portions, a curved portion of each tongue extending into the central cavity of the hollow body at a similar or the same height to create a channel therebetween, the channel being narrower than the width of the central cavity of the hollow body.

In further examples the retaining portions comprises a wedge-shaped slot in the hollow body wall.

In further examples the wedge-shaped slot comprises a broader end for guiding the packaging into the slot and an opposing narrower end for securing the packaging or wedging the packaging thereinto.

In further examples the holding devices comprises one or more attachment members for engaging suction tubing designed for coupling to the medical equipment.

In further examples the one or more attachment members may include two cylindrical protrusions located on opposite sides of the holding device.

In further examples the holding device comprising a tubing retention mechanism for retaining the suction tubing coiled in a coiled state.

In further examples the tubing retention mechanism is formed from a pocket, hook, flange, shelf or ridge.

In further examples the mounting component is the holding device body.

In further examples the mounting component is a mounting plate directly or indirectly connected to the holding device body.

In further examples the mounting plate is substantially planar and includes one or means for mounting such as adhesive, screw apertures, flanges, clips, hooks or interference fit mechanisms.

In further examples the mounting plate is curved and includes one or more means for mounting such as adhesive, screw apertures, flanges, clips, hooks or interference fit mechanisms.

In further examples the mounting component is a mounting arm directly or indirectly connected to the holding device body.

In further examples the mounting arm comprises a clasping or clipping means for engagement with an object.

In a further aspect of the invention there is provided a method for separating medical equipment from associated medical equipment packaging, the method comprising;
inserting medical equipment retained in unopened packaging into the body of a holding device having a body and retaining portions;
causing the packaging to be configured from an unopened state to an opened state;
inserting at least a portion of the opened state packaging into the retaining portions;
removing the medical equipment from the holding device, such that upon or during removal of the medical equipment from the holding device, the opened packaging is retained in the holding device by the retaining portions and the medical equipment becomes separated from the packaging.

According to a still further aspect of the invention, there is provided a method of holding or storing a medical suction system comprising a packaged suction tip and suction tube, the method comprising;
mounting a holding device for medical equipment as described above to a surface;
placing the suction tip in the body of the holding device;
placing a portion of the suction tip packaging in the one or more retaining portions of the holding device.

In examples the medical equipment is Yankauer sucker tip.

### BRIEF DESCRIPTION OF THE DRAWINGS

FIGS. 1A-1D shows a back view, perspective view, front view and side view of one example of a holding device;

FIG. 2 shows a suction tip apparatus in a packaging sleeve in a closed configuration;

FIG. 3 shows a holding device in a further example with a suction tip apparatus in closed packaging and suction canister;

FIG. 4A shows an alternative example of a holding device with a curved mounting place for attachment to a suction canister;

FIG. 4B shows a suction tip apparatus in partially opened packaging, retained in one example of the holding device;

FIGS. 5A and 5B show the holding device in an alternative example of the invention;

FIG. 6 shows one example of a mounting plate for use with the holding device of the present invention;

FIG. 7 shows a backing plate for use with the holding device of FIGS. 5A, 5B and 6 in one example of the invention;

FIGS. 8A-C shows a holding device and mounting plate suitable for attachment to a canister in a further example of the invention;

FIGS. 9A-D show a holding device and mounting plate suitable for use in a various clinical environments in a further example of the invention;

FIGS. 10A-C shows a holding device and mounting assembly suitable for use when attached to a medical rail in a further example of the invention;

FIG. 11A-C show a holding device and mounting plate suitable for use in environments such as hospital wards in a further example of the invention; and

FIG. 12 shows a hollow body and retaining portion of the holding device in a further example of the invention.

### DETAILED DESCRIPTION OF THE EMBODIMENTS

Described herein is a holding device for holding a medical apparatus. The medical apparatus may be a suction apparatus, such as the front end or the tip of a Yankauer sucker or a Yankauer suction catheter. The specification below describes and illustrates a holding device in the context of holding such a suction apparatus, but it should be apparent to a skilled person in the art that holding device 100 is capable of holding a range of different medical apparatus.

In medical environments, the use of a suction apparatus, such as the well-known Yankeur suction device, often occurs in situations of high pressure and time sensitivity. The ability to quickly access sterilised or very clean equipment can lead to improved patient outcomes and a more streamlined process.

Suction equipment typically includes a suction tip attached to a length of tubing, which is itself attached to a suction cannister for both providing suction and receiving fluids. New and/or sterilised suction tips often come in packaging formed from two sheets of packaging sealed together to create a bag, sleeve or envelope around the suction tip. In use, the yankeur suction tip is held within the holding device, the two parts of the packaging separated on opening to access the suction tip and the two parts of the packaging are retained by the holding device to ensure swift and unobstructed removal of the suction tip for use with a patient.

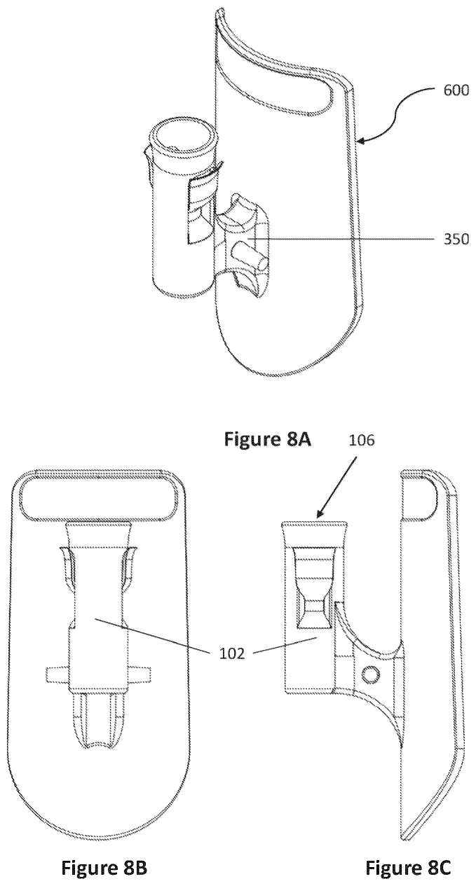

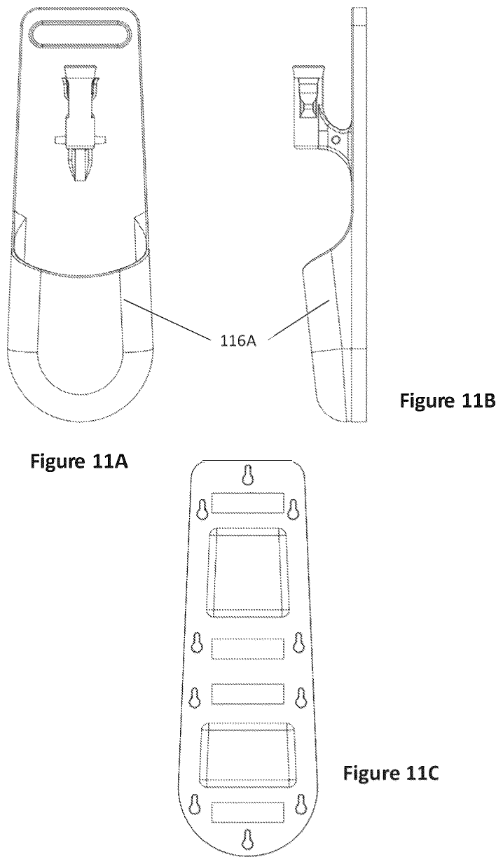

The holding device may be formed with a range of different mounting configurations to suit different medical environments. For example, the wall-mountable holding device as exemplified in FIGS. 11A-C may be secured on a wall in a hospital ward or on any suitable vertical or substantially vertical surface, while that shown in FIGS. 8A-8C has a curved mounting plate, allowing for direct attachment to a suction collection cannister or other curved surface.

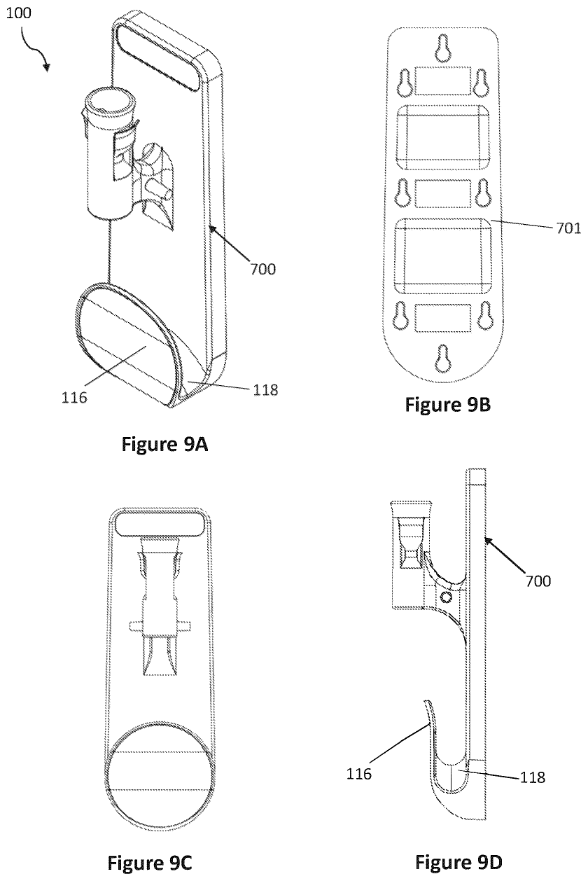

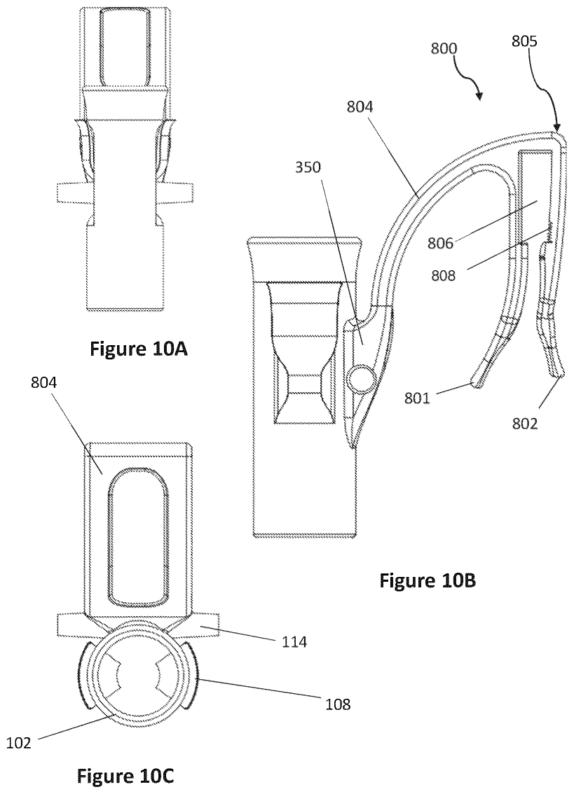

A further variation as shown in FIGS. 10A-C may be used during a medical procedure where the holding device may be clipped or hooked on a medical rail for ease of access during the procedure and FIGS. 9A-D provide a still further example of a holding device suitable for use in a various clinical environments, for example a rapid response environment.

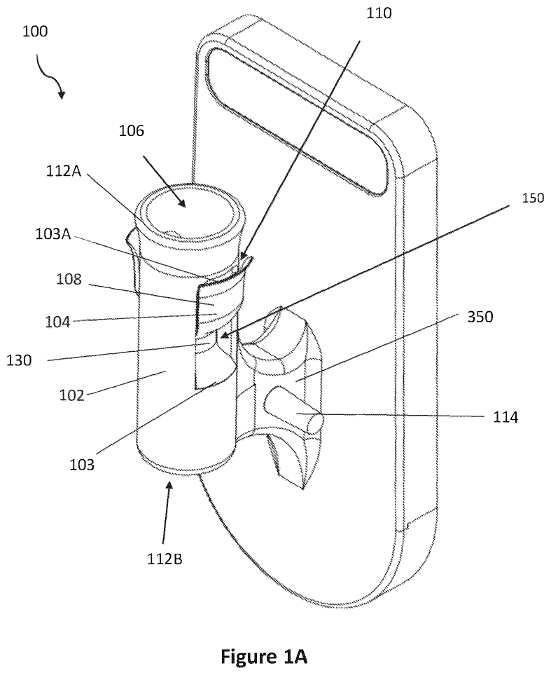

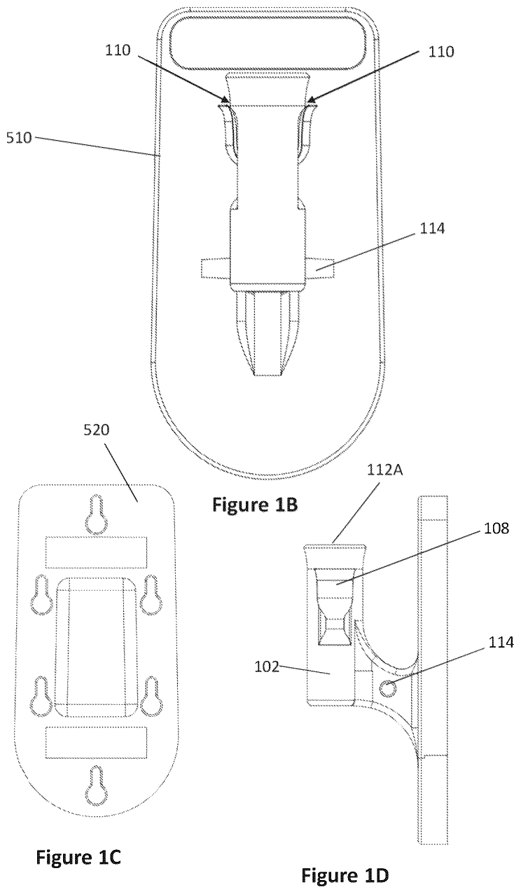

FIGS. 1A-1D illustrate different views of an embodiment of a holding device 100.

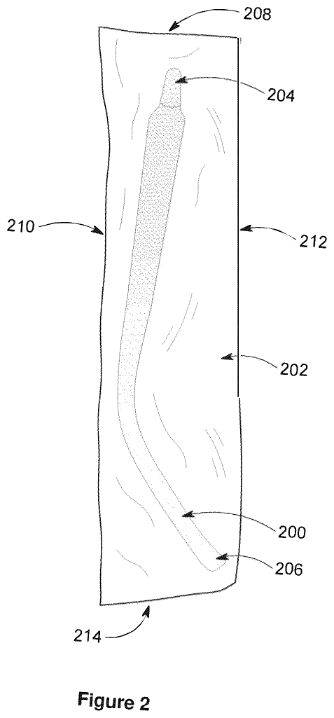

Before describing holding device 100 in further detail, an example of a suction apparatus suited to being held by holding device 100 is now described. FIG. 2 illustrates a suction apparatus 200 packaged in its unopened packaging. Suction apparatus 200 is the front end or the suction tip of a suction system such as a Yankauer sucker or a generic suction catheter. Suction apparatus 200 may be made of plastic and for single use, or may be stainless steel that may be re-used and re-packaged after sterilisation. A first end 204 of suction apparatus 200 is adapted to be connected to suction tubing, which in turn is connected to the suction-holding canister for collecting any contents from suctioning. A second end 206 of suction apparatus 200 is adapted to be placed in a patient's oral cavity to provide suction. As illustrated in FIG. 2, suction apparatus 200 includes two relatively straight sections connected by an intermediate curved portion, resulting in an overall curved suction apparatus. With the suction tubing and the suction-holding canister appropriately connected to the suction system, suction apparatus 200 may provide suction at second end 206 for removing fluids in the patient's oral cavity and/or upper airway for example.

The packaging of suction apparatus 200 is in the form of a packaging sleeve 202.

Packaging sleeve 202 includes a front sheet and a back sheet that are sealed along or near their edges for enclosing the suction apparatus. Packaging sleeve 202 includes top edge 208 (corresponding to first end 204 of suction apparatus 200), two side edges 210 and 212, and bottom edge 214 (corresponding to end 206 of suction apparatus 200).

The front sheet may be clear or semi-transparent, while the back sheet may be opaque. Packaging sleeve 202 is be configurable from an unopened state to an opened state by separating the front and back packaging sheets from each other (opening the packaging). As discussed in the Background, packaging is sometimes pre-opened long before the contents are used to provide quick access to the suction apparatus. Opening the packaging may be achieved by, akin to peeling a banana, breaking the seal along a first edge (e.g. edge 208) of the packaging sleeve and separating the front sheet and the back sheet by further breaking the seals along the two edges adjacent to the first edge (e.g. side edges 210 and 212).

Referring to FIGS. 1A-1D , holding device 100 comprises a body and retaining portions 104. In one arrangement, the body is a hollow body 102 having a tubular form and defines a region of empty space 106. Hollow body 102 is designed to receive a packaged suction tip (such as that shown in FIG. 2) into empty space 106. The packaging may be unopened or opened. When formed as a cylinder, hollow body 102 may have a portion of the inner diameter sized to receive and retain a packaged medical apparatus. In one example this portion may be slightly less than the diameter of a packaged medical apparatus when placed in the holding device.

In examples, a portion of the inner diameter of hollow body 102 is a narrow region that is less than the full width of a packaged suction apparatus so as to accommodate the suction apparatus in the packaging and have to held within the hollow body. Where the narrow region is less than the diameter of the packaging, the unopened suction apparatus can be retained through a friction fit between the packaging and the narrow region. The narrow region may be created by shaped inner walls of the hollow body 102, or by opposing sections of the retaining portions 104 that are curved an extend partially into the cavity of the hollow body. In preferred examples the narrow region is between 10 mm-25 mm, more preferably 15 mm-20 mm and in one specific preferred embodiment, substantially 16 mm. These sizes are not intended to be limiting.

In alternative examples, inner wall of hollow body 102 may include flanges, ribs, clasps or regions of curved surface to provide a means to reduce the diameter of the hollow body at one or more points along the length of the hollow body.

Hollow body 102 may be open at a first end 112A and at an opposed second end 112B. Open-ended hollow body 102 may be shorter than the suction apparatus which holding device 100 is designed to hold, so that portions of the suction apparatus may protrude from both the first end 112A and the second end 112B when inserted in holding device 100. A portion of the suction apparatus protruding from first end 112A may provide a handle for easy access of the suction apparatus. In case of a curved suction apparatus, such as the Yankauer sucker tip shown in FIG. 2, hollow body 102 is sized in length to accommodate the curvature of the curved suction apparatus.

In alternative examples, the body of the holder is in the form of one or more clips or brackets adapted to retain a suction tip apparatus, or is a hollow body having a square, oval, rectangular or other polygonal or irregular polygonal cross section.

Retaining portion 104 of holding device 100 is adapted to retain a suction apparatus' open packaging within hollow body 102. In one example, retaining portion 104 is formed integrally with the hollow body 102. For example, holding device 100 may be made of plastic, and retaining portions 104 may be formed in the same injection moulding or additive manufacturing process as the hollow body 102. In alternative examples not shown, the retaining portions 104 may be formed separately from the hollow body 102 and be connected to the exterior of hollow body 102. In still further examples, the retaining portions 104 and body 102 may be each connected to a single/the same mounting member, but not directly connected to each other.

The retaining portion may take the form of a groove, slot, channel, hook or gripping means formed in, or connected to body 102.

In the example shown in FIG. 1A, hollow body 102 includes two opposing apertures 150 formed in the opposing side walls of hollow body 102. Apertures 150 may be rectangular, other polygonal or irregular shape, including wedge shaped with the wedge having a wider opening towards first end 112A of hollow body 102 compared to an opposing bottom end. The aperture may extend between 10%-90% of the length of the hollow body, or in preferred examples between 40%-60% of the length of the hollow body, the aperture size providing a length to allow the insertion and retention of packaging within the retaining portions.

Retaining portions 104 comprise a tongue 108 extending from a proximal end 103 adjacent hollow body 102 to a vertically opposing distal end 103A spaced away from and unattached to hollow body 102, the distance between the hollow body 102 and distal end 103A defined by opening 110. In alternative embodiments envisaged but not exemplified the proximal and distal ends of tongue 108 are substantially horizontally opposed from each other, creating a tongue portion wherein in use, the packaging is inserted between the tongue and hollow body in a sideways motion, from an unattached distal end and pulled horizontally towards the opposing proximal end of the tongue.

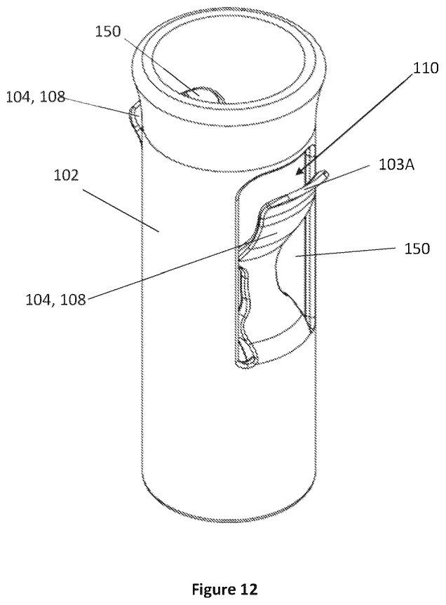

In preferred examples shown, tongue 108 is at least partially located within aperture 150 and defines an opening 110 between the distal end 103A of tongue 108 and hollow body 102 for receiving portions of the opened packaging in a downward motion. In another arrangement, to assist retaining the packaging, the opening 110 may be sized to be comparable to the thickness of the front sheet or the back sheet of the packaging, such that a substantial portion of opening 110 contributes to a retaining action. In other examples, opening 110 may be wider than the thickness of the packaging to increase ease of entry of packaging into the opening, with the remaining tongue portions having a retaining function by creating a fractioned path for the packaging to enter and remain. An example of this may be seen in FIG. 12. In some examples, the distance between the hollow body 102 and distal end 103A of tongue 108 may be 2 mm-20 mm for example, preferably 2 mm-10 mm.

At its distal end 103A, tongue 108 may comprise a flanged and/or curved tip for facilitating or guiding the packaging to be received into opening 110 and aperture 150. Tongue 108 may be so tapered that the distal end of tongue 108 is wider than the proximal end of tongue 108 or vice versa.

In further embodiments as seen in multiple figures, tongue 108 may include a central taper 130, creating a “waist” to tongue 108. The shaping of tongue 108 to comprise a central taper 130 results in the formation of multiple points of decreased space between the tongue edges and the edge of aperture 150, creating pinch points for the packaging to be wedged or held in. In other embodiments, the gap between the tongue edges and the edge of aperture 150 is large enough so that pinch points aren't created, but will provide space to allow packaging pushed into this gap to scrunch up and be retained through friction forces provided by the tongue and edges of aperture 150.

Tongue 108 may be formed with one or more curves across the horizontal axis and/or vertical axis of the tongue. In the embodiment seen in at least FIGS. 1A-1D , the distal end of the tongue 103A curves outwardly forming a lip to aid in guiding the packaging into the slot formed between the tongue 108 and body 102. Tongue 108 is curved both inwardly around a horizontal axis towards the centre of hollow body 102, such that a portion of the tongue surrounding central portion 130 is received within the cavity of the hollow body 102. Opposing regions 130 extending into the body cavity provides two opposing surfaces that reduce the space within the hollow body and act to hold the suction tip in an upright position, both when the packaging is in an open and closed configuration. This is particularly useful when the packaging is unopened, as the user can then open the packaging with the suction tip held in the correct position by the holding device.

The attachment of tongues 108 to the body at one end 103 imparts flexibility to the tongue portions. When a suction tip apparatus is inserted within the central cavity 106, a force is imparted against each of the central tapered portions 130 of the tongues that extend into the central cavity 106 by the packaged apparatus. The tongues may flex around the connection point with the hollow body, but bias back towards the original position, providing an additional force to retain the suction apparatus within central cavity 106.

Tongue 108 is also preferably curved around a vertical axis between the proximal and distal ends of the tongue to create a tongue having a curved wall similar to the curvature of the wall of hollow body 102. Tapered region 130 of the hollow body 102 may extend at least partially through aperture 150 into the cavity of hollow body 102. The addition of curves or tapers on the tongue aids in guiding the packing into opening 110 and securely retaining the packaging within opening 110. The number and size of curves in the tongue will dictate the space between the tongue and holding device body 102, in turn influencing how securely the packaging is held in position. It is envisaged that the specific dimensions can change depending on the type of packaging and expected force to be imparted on the tongue when the suction or other medical device is removed from the packaging. FIGS. 1A and 12 for example show slightly different configurations and sizing of retaining portions 104 compared to body 102.

Tongue 108 is preferably formed from a smooth surface to guide the packing along the inside face of the tongue towards the base of the tongue, but may include areas of textured surface or protrusions to aid in packaging retention by preventing movement of the packaging once inserted.

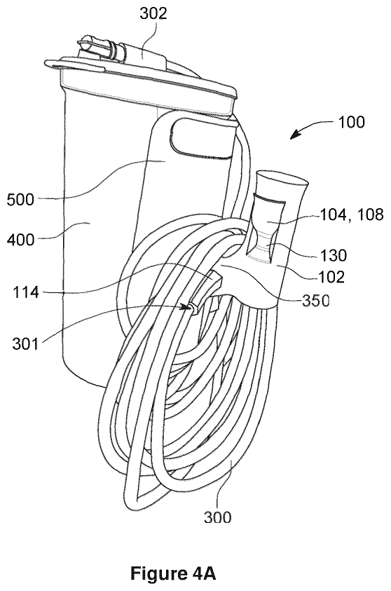

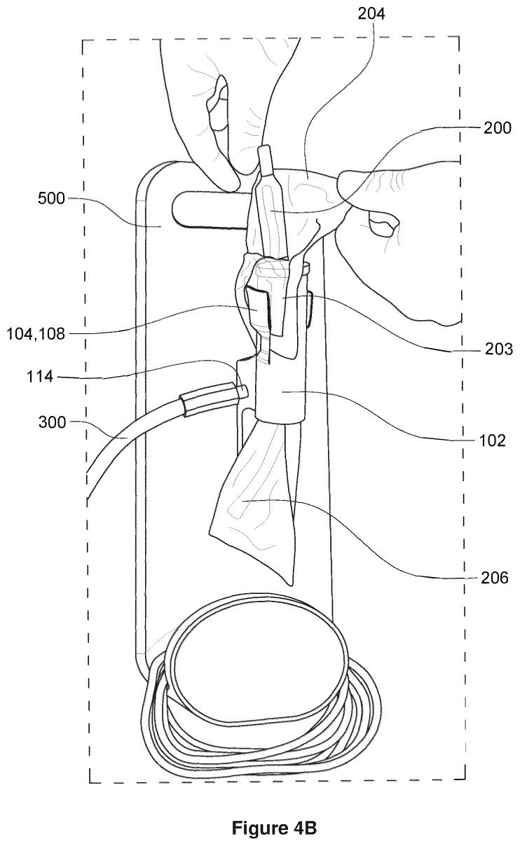

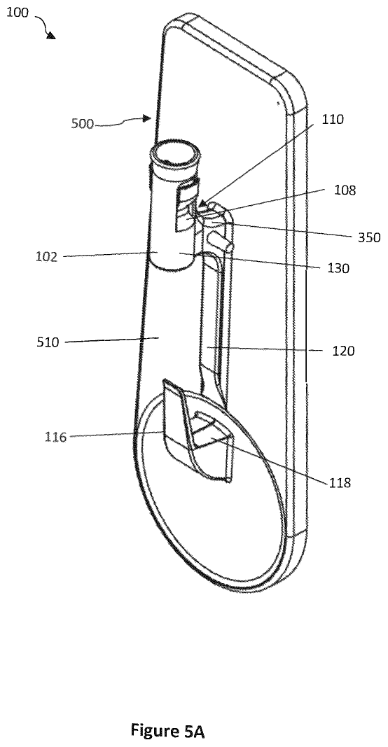

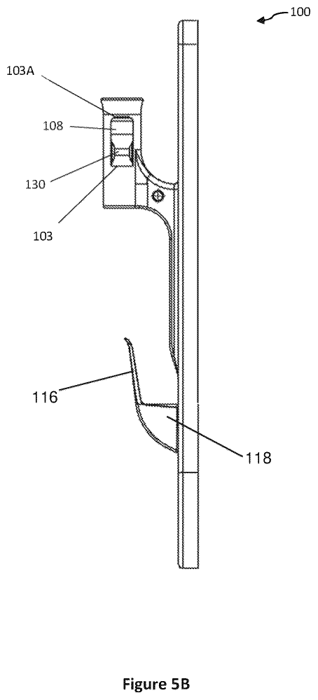

Hollow body 102 of FIGS. 1A-1D is connected to neck portion 350. Neck portion 350 retains holding device body 102 in a substantially vertical position and provides a means for connection to a mounting plate or other mounting assembly. The size and shape of neck 350 may change depending on the mounting requirements. Neck portion 350 further provides a region to support suction tubing as seen in FIG. 4A.

Accordingly, with a suction apparatus in its packaging inserted in holding device 100 and the packaging received or wedged into opening 110, retaining portions 104 may hold the packaging in place (i.e. in hollow body 102) if and when the suction apparatus is removed from the opened packaging from the holding device 100. This retaining action may be necessary where the suction apparatus is quickly removed from holding device 100 (due to, for example, an emergency) with a tendency to also dislodge the packaging. The retaining action is sufficient to retain the opened packaging while the suction apparatus is providing suction and while suction apparatus is being removed from the opened packaging. The retention of the packaging also facilitates a one-handed operation of the suction device, which may be crucial in an emergency situation.

In further possible embodiments of the invention envisaged but not exemplified, the retention means may be in the form of clamps, clips, rings, clasps or other friction fit mechanisms capable of receiving and retaining packaging when withdrawing the suction apparatus.

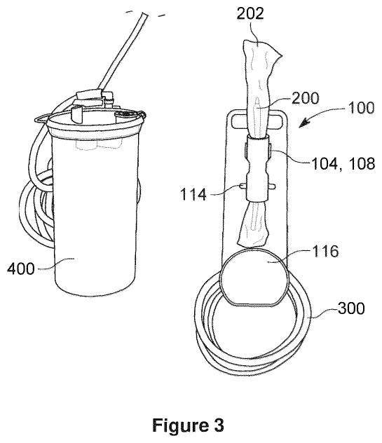

The suction apparatus is designed to be used in conjunction with the suction tubing (see 300 in, for example, FIG. 3), made in some cases from a flexible plastic material, designed for coupling to the suction apparatus. Referring to FIGS. 1A-D, and 6-11 inclusive holding device 100 may include features to arrange the suction tubing for quick access. For example, holding device 100 may further comprise one or more nipples 114 attached to neck portion 350 for engaging the suction tubing, preferably extending substantially horizontally from the neck portion. Nipple 114 may take the form of a cylindrical protrusion or wedge having an outer diameter that is substantially equal to or slightly larger than the inner diameter of the suction tubing at a first end connected to the apparatus and a diameter smaller that the inner diameter of the suction tubing at an opposing second end, with a gradient therebetween. This allows the suction tubing 300, which may slightly stretch or expand radially upon engaging nipple 114, to be easily received on the smaller second end of nipple 114 and securely held in place once pushed further towards the wider first end, providing quicker or more convenient access by hospital staff during urgent use.

Further, holding device 100 may have a tubing holding device 116 for holding the suction tubing coiled in a coiled state, seen in examples shown in FIGS. 5A-5B, 9A-D and 11A-C. Tubing holding device 116 may include a bent portion 118 for confining the suction tubing coiled in a coiled state (FIGS. 9A-D). Bent portion 118 may be flexible. In one arrangement, bent portion 118 may be resiliently flexible, in which case the coiled suction tubing may be quickly pulled from holding device 100 without the need to lift the suction tubing off tubing holding device 116. The resiliently flexible bent portion therefore facilitates retrieval of the confined suction tubing from tubing holding device 116. Tubing holding device 116 may be connected to hollow body 102 via a rigid neck portion 120. The neck portion may be flat on one side where a fastener, such as screws or adhesive, may be applied to fasten holding device 100 on, for example, a corresponding flat surface such as a wall.

FIGS. 3 to 4B illustrate a number of possible uses of holding device 100 in conjunction with suction apparatus 200 and its packaging sleeve 202. FIG. 3 illustrates wall-mounted holding device 100 in one embodiment together with a suction apparatus 200 inserted in its unopened packaging sleeve 220. The retaining portions may include a plurality of openings. For example, holding device 100 has two openings 110 located on opposite sides of hollow body 102, each opening defined by its corresponding tongue 108 (more clearly seen in FIGS. 1, 6A-11D). The two oppositely located openings 110 may be adapted for receiving, respectively, the front sheet and the back sheet of the packaging.

As illustrated in FIG. 3, the suction apparatus has been placed and received in hollow body 102. In this use of the suction apparatus 200, packaging sleeve 202 may remain unopened to ensure that suction apparatus 200 stays sterile. An advantage of holding device 100 in this use may therefore be to provide a designated place for placing suction apparatus 200. This may assist hospital staff to quickly locate suction apparatus 200 if and when it is required.

As illustrated in FIGS. 3 and 4A, holding device 100 may be located near or be curved to mount directly on a suction canister 400. Suction tubing 300 may be arranged in a coiled state around suction tubing holding device 116 (FIG. 3) or neck 350 (FIG. 4A). As in FIGS. 3 and 4A, one end 301 end of suction tubing 300 adapted to couple to suction apparatus 200 (to conduct solids or fluids collected by suction apparatus 200) may be engaged to nipple 114 when not in use. The other end 302 of suction tubing 300 may be coupled to suction canister 400. Holding device 100 is located close to the suction canister 400 so that, while holding device 100 provides support to hold suction tubing 300, assembly of the suction system is substantially complete to provide quick access to suction if and when required.

To provide even quicker access to suction apparatus 200, packaging sleeve 202 may be pre-opened. FIG. 4B shows packaging 202 being opened, with the front sheet 203 inserted into retaining means 104, and backing sheet 204 held in position ready to be, but not yet inserted into retaining means 104. Suction apparatus 200 may remain in the opened packaging within hollow body 102, with the end 206 of suction apparatus 200 remaining within a closed portion of the packaging to minimise exposure to contamination. To reduce the chance of contamination, the packaging may be opened along top edge 208 (corresponding to end 204 of suction apparatus 200) rather than the bottom edge 214 (corresponding to end 206 of suction apparatus 200). The front sheet and the back sheet of the opened packaging sleeve 202 are peeled back and, respectively, received into two oppositely located openings 110 and then pulled further in a downward fashion towards the base of tongue 108. The minimal space between the edges of tongue 108 and the edges of aperture 150 and the inwardly curved shape of tongue 108 imparting a friction force on the packaging, retaining it in position.

Additionally, or alternatively, openings 110 may each have a gap comparable to the thickness of the front sheet or the back sheet of packaging. In some examples, the distance between the tongue 108 and aperture 150 may be narrower than the sheet thickness in particular regions so that the tongue may flex slightly away from body 102 on insertion of the packaging sheet, the biasing force towards the body 102 helping to securely retain the packaging. Opening 110 is located towards top end 112A of hollow body 102 such that, when the opened packaging is received into opening 110, a handle or a portion of the suction apparatus 200 protrudes out of hollow body 102.

When suction is required, a suction apparatus 200 can be lifted and removed from the packaging, leaving the packaging retained in hollow body 102. Apparatus 200 may then be connected with suction tubing 300 after disengaging suction tubing 300 from nipple 114. The suction system may then be activated to provide suction.

To further improve the access speed of the suction system, suction apparatus 200, suction tubing 300 and suction canister 400 may be connected and be ready to provide suction upon activation of the suction system. In some cases, suction tubing 300 may have a degree of resilience and may therefore assert some pulling tension tending to dislodge suction apparatus 200 from holding device 100. To avoid dislodgement, a portion of the inner diameter of hollow body 102 is designed to be comparable and slightly narrower than suction apparatus 200 inserted in its packaging, such that suction apparatus 200 when received by hollow body 102 is maintained substantially vertically rather than at an angle. In this case, suction apparatus 200 is caused to press via the packaging against an inner surface of hollow body 102 and tapered portions 130 of tongues 108.

When suction is required from an arrangement illustrated in FIG. 3, two modes of access are possible. In the first mode of access, suction apparatus 200 may be lifted and removed from the packaging, leaving the packaging retained in hollow body 102 by retaining portions 104.

The suction system may then be activated to provide suction. This mode corresponds to a two-hand operation since once suction apparatus 200 is lifted by one hand, another hand is needed to activate the suction system. In the second mode of access, the suction system may first be activated to provide suction. Suction apparatus 200 may then be lifted and removed from the packaging, leaving the packaging retained in hollow body 102 by retaining portions 104. This mode corresponds to a one-hand operation.

The possibility of the one-hand operation illustrates a function of retaining portions 104. When the suction system is activated before suction apparatus 200 is removed from its packaging sleeve 202, suction may cause suction apparatus 200 to be attached or otherwise bound to packaging sleeve 202. If no retaining portions 104 are present to retain the packaging in hollow body 102, lifting suction apparatus 200 is likely to result in also lifting packaging sleeve 202, ultimately requiring another hand to retain or separate packaging sleeving 202 from suction apparatus 200.

As shown in FIGS. 5A and 5B, holding device 100 may be directly or indirectly (for example via a wall bracket) mounted in a vertical orientation to a wall, via rigid neck portion 350, such that bent portion 118 used to support suction tubing is positioned below hollow body 102.

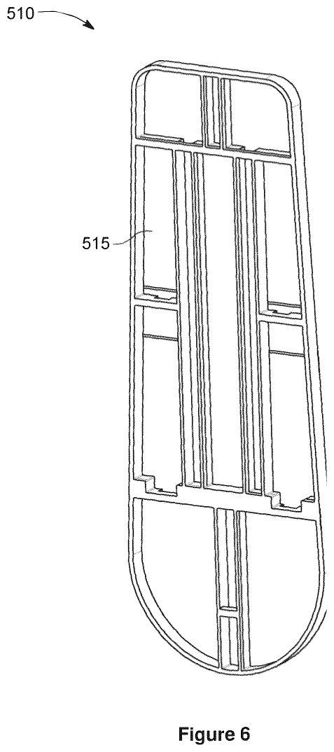

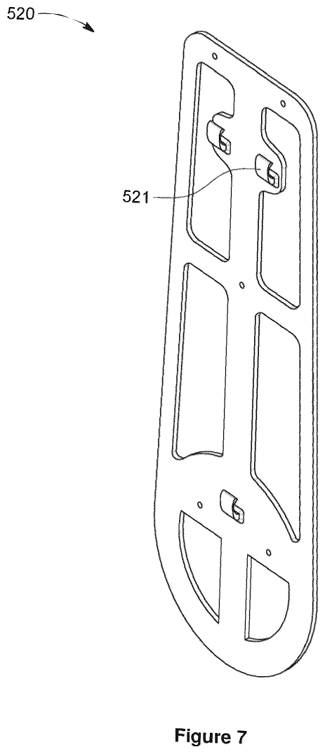

An alternative mounting arrangement of holding device 100 is shown in FIGS. 5A, 5B, 6 and FIG. 7 wherein holding device 100 includes mounting plate 500. Holding device 100 is preferably integrally formed with mounting plate 500 but may also be constructed as a separate piece (for example comprising a rigid neck portion 350) that can be attached to a mounting plate as needed. In the example shown in FIGS. 5A, 5B and 6, mounting plate 500 comprises a front plate 510, integrally formed as part of holding device 100, FIG. 5A showing the front face of front plate 510 on which hollow body 102 is mounted and FIG. 6 showing the opposing rear face 515 of front plate 510, rear face 515 adapted for connection to removable backing plate 520, as shown in FIG. 7. Backing plate 520 may be screwed or otherwise mounted to a wall, trolley or other item of furniture as required, with front plate 510 connectable via rear face 515 to backing plate 520 by hooks 521. In other embodiments front plate 510 and backing plate 520 may connect using friction fit connections, clamps, screws or other known connection means. FIGS. 1A-1D also show a variation with front plate 510 and backing plate 520, allowing wall mounting of holding device 100.

Backing plates 520 may be formed with a range of different connection mechanisms to suit the environment in which the device it to be used, for example permanent locations such as hospital emergency rooms or wards, dental or veterinary clinics where wall mounting is likely, or in more temporary surroundings or locations such as hospitals beds, ceiling mounted pendulums or other hospital equipment (for example an anaesthetic machine), ambulances or temporary medical environments where mounting via a hook or clamp may be more practical.

Alternatively, mounting plate 500 may be a single piece construction that is mounted or connected directly to a structure for use. The shape of the mounting plate 500 may be altered to suit attachment to surfaces and apparatus of different shapes, including cylinders, bed rails or bars, walls, cabinetry or shelving for example that may be found across a range of different environments.

A range of different holding device examples suitable for mounting/connection to different objects or surfaces are shown in FIGS. 8A-11C. In the examples provided, the holding device mechanism remains the same, but may be mounted in a variety of different ways.

FIGS. 8A-8C include a holding device 100 attached to curved mounting plate 600. Holding device 100 is preferably formed with a mounting plate 600 is curved around a longitudinal axis and is designed for direct attachment to a suction cannister or other curved surface. Attachment to the cannister may be by known means such as adhesive, straps, hooks or other surface mounting means. The arc of the curve may be tailored to suit particular cannister shapes or sizes as required.

Holding device 100 connects to mounting plate 600 via neck portion 350.

FIGS. 9A-9D provide a further example of a holding device attached to a mounting unit suitable for use in various clinical environments such as an ambulance or other rapid response environments. As with the example provided in FIGS. 9A-9C, a tubing holding device 116 is located beneath the holding device 100 and extends outwardly from mounting plate 700, providing a bent portion 118 for holding the suction tubing in a similar fashion to that shown in FIG. 4. Backing plate 701 may be used to wall mount the device to the wall, although this is not intended to be limiting and other known mounting techniques may be used.

FIGS. 10A-10C show a further example of a device 800 including a holding device 100 with a mounting arm 804 extending from neck portion 350. The use of mounting arm 804 allows for the holding device to be clipped to a medical rail such as found on a ventilator machine, operating table or ceiling mounted moveable pendulum. In the example provided, mounting arm 804 extends upwardly from the rear of holding device 100 curving away from holding device 100. To enable attachment to the rail of an operating table (or shelf, trolley, medical pendulums, ventilators, or other transportable storage system in other environments), clasp 805 extends approximately perpendicular to the distal end of arm 804 and is formed by two opposing members 801 and 802, defining a space 806 therebetween. Space 806 may vary in size to accommodate different sized rails. To mount the device 800, for example on a rail, the rail is positioned between members 801 and 802 and a downward force applied to arm 804, forcing the rail into space 806. Members 801 and 802 are preferably formed from resilient material, such that when forced apart, the members 801 and 802 are biased together, helping maintain the device 800 to be mounted on the rail. Additional features to increase the mounting stability may be added, such as teeth 808 formed on the walls of members 801 and 802. It is envisaged that the arm and clasping members may alter in size and shape to suit different mounting requirements, while retaining the core holding device features.

In further variation of a wall mounted holding device is seen in FIGS. 11A-11C. In this example, tube holding device 116 is in the form of a pocket 116A with an upward facing open end, into which suction tubing can be folded away inside. The pocket and subsequent mounting plate may vary in size depending on the size and length of tubing it is required to hold.

Now that arrangements of a holding device for holding a medical apparatus have been described, it should be apparent to a skilled person in the art that the described arrangements include the following advantages:

The holding device may be used to provide a designated location for locating a medical apparatus. In applying to a suction system, the holding device if located near a suction canister and suction outlet may allow a substantially complete system to be set up for quick access to suction.

In applying to a suction system, the holding device may be used to organise tubing for allowing quick access to suction.

In applying to a suction system, the holding device may be used in an arrangement to allow one-hand operation.

The holding device may be mounted in a variety of different ways to suit varied medical, dental and veterinary environments.

It will be understood that the invention disclosed and defined in this specification extends to all alternative combinations of two or more of the individual features mentioned or evident from the text or drawings. All of these different combinations constitute various alternative aspects of the invention.

1-31. (canceled)

32. A holding device for supporting medical equipment encased or partially encased in packaging, the packaging being configurable from an unopened state to an opened state, the holding device comprising;
a hollow body formed from one or more body walls defining a central cavity, the central cavity shaped to receive and hold the medical equipment;
at least one retaining portion; and
a mounting component;
wherein the at least one retaining portion is adapted to retain at least a portion of the packaging when the packaging is configured to the opened state, such that upon or during removal of the medical equipment from the holding device and/or the opened packaging, the packaging remains retained or partially retained by the at least one retaining portion of the holding device.

33. The holding device of claim 32, wherein the body is a cylindrical hollow body, open at a first end and optionally at an opposing second end.

34. The holding device of any one of claim 32, wherein the at least one retaining portion is formed in or on the one or more walls of the hollow body.

35. The holding device of claim 34, wherein the holding device comprises two retaining portions positioned on opposing sides of the hollow body.

36. The holding device of claim 32, wherein the hollow body is shaped to define at least one aperture in the body wall, the aperture defining a space that surrounds at least a portion of an at least one retaining portion.

37. The holding device of claim 36, wherein the holding device is configured to define two opposing elongate apertures in the body walls, the apertures extending between 10%-90% of the length of the hollow body.

38. The holding device of claim 36, wherein the at least one retaining portion comprises a tongue having a top end and an opposing base end, the tongue extending at least partially outwardly from the hollow body wall at a top end of the tongue to define a space or gap between the top end of the tongue and hollow body wall, the tongue directly or indirectly mounted to the hollow body wall at a base end of the tongue.

39. The holding device of claim 36, wherein the at least one aperture surrounds or partially surrounds at least one tongue, the at least one tongue directly or indirectly connected to the hollow body at a/the base end of the at least one tongue.

40. The holding device of claim 39, wherein the at least one tongue comprises a flanged and/or curved tip at the top end of the at least one tongue.

41. The holding device of claim 40, wherein the at least one tongue is tapered or curved at one or more regions or along one or more axis.

42. The holding device of claim 41, wherein the at least one tongue is curved such that a portion of the curved tongue extends through the aperture in the body wall into the central cavity of the hollow body.

43. The holding device of claim 42, wherein the holding device includes two opposing tongue portions, a curved portion of each tongue extending into the central cavity of the hollow body to create a channel therebetween, the channel being narrower than the width of the central cavity of the hollow body.

44. The holding device of claim 34, wherein the at least one retaining portion is formed from a wedge-shaped slot in the hollow body wall, the wedge-shaped slot comprising a broader end for guiding the packaging into the slot and an opposing narrower end for securing the packaging or wedging the packaging thereinto.

45. The holding device of claim 32 comprising one or more attachment members for engaging suction tubing, the one or more attachment members comprising two cylindrical protrusions located on opposite sides of the holding device.

46. The holding device of claim 32, the holding device comprising a tubing retention mechanism for retaining the suction tubing in a coiled state.

47. The holding device of claim 32, wherein the mounting component is the holding device body.

48. The holding device claim 32, wherein the mounting component is a mounting plate directly or indirectly connected to the holding device body.

49. The holding device of claim 48, wherein the mounting plate is curved and includes one or more means for mounting such as adhesive, screw apertures, flanges, clips, hooks or interference fit mechanisms.

50. The holding device of claim 32, wherein the mounting component is a mounting arm directly or indirectly connected to the holding device body, the mounting arm comprising a clasping or clipping means for engagement with an object.

51. A holding device for supporting a component of a medical suction system encased or partially encased in packaging, the packaging being configurable from an unopened state to an opened state, the holding device comprising;
a cylindrical hollow body defining a central cavity, the central cavity shaped to receive and hold a packaged suction tip of a suction system;
at least one retaining portion formed in or on the cylindrical hollow body, the at least one retaining portion configured to retain at least a portion of the packaging of the suction tip of the suction system when the packaging is in an opened state; and
a mounting component for mounting the holding device to a surface.
